(19) United States
(12) Patent Application Publication
(10) Pub. No.: US 2011/0086103 A1
(43) Pub. Date: Apr. 14, 2011

(54) NOVEL MANDELATE SALT OF FESOTERODINE

(75) Inventors: Kishore Charugundla, Andhra Pradesh (IN); Udhaya Kumar, Pondicherry (IN); Praveen Kumar Neela, Andhra Pradesh (IN); Nitin Sharadchandra Pradhan, Maharashtra (IN); Jon Valgeirsson, Hafnarfjorour (IS)

(73) Assignee: ACTAVIS GROUP PTC EHF, Hafnarfjordur (IS)

(21) Appl. No.: 12/936,241

(22) PCT Filed: Apr. 6, 2009
(86) PCT No.: PCT/IB2009/005679

§ 371 Date: Dec. 13, 2010

(30) Foreign Application Priority Data
Apr. 4, 2008 (IN) 862/CHE/2008

(51) Int. Cl. A61K 9/14; C07C 69/00; A61K 31/24; A61P 1/00; A61P 13/00

(52) U.S. Cl. 424/489; 560/140; 514/546

(57) ABSTRACT

Provided herein is a novel raantlelate sail of fesoterodine, process for the preparation, pharmaceutics!! compositions, and method of treating thereof. Provided also herein are solid state forms of fesoterodine mandelate, process for the preparation, pharmaceutical compositions, and method of treating thereof. The raandelate salt of fesoterodine is useful for preparing fesoterodine free base or a pharmaceutically acceptable salt thereof; particularly fesoterodine fumaraie, in high purity.

### CROSS REFERENCE TO RELATED APPLICATION

This application claims the benefit of priority to Indian provisional application No. 862/CHE/2008, filed on Apr. 4, 2008, which is incorporated herein by reference in its entirety.

### FIELD OF THE DISCLOSURE

The present disclosure relates to novel salts of fesoterodine, process for preparation, pharmaceutical compositions, and method of treating thereof.

### BACKGROUND

U.S. Pat. No. 6,713,464 discloses a variety of 3,3-diphenylpropylamine derivatives, processes for their preparation, pharmaceutical compositions comprising them, and method of use thereof. These compounds are antimuscarinic agents with superior pharmacokinetic properties compared to existing; drugs such as oxybutynin and tolterodine and useful in the treatment of urinary incontinence, gastrointestinal hyperactivity (irritable bowel syndrome) and other smooth muscle contractile conditions. Among them, Fesoterodine, 210R)-3-[bis(1-methylethyl)amino-1-phenylpropyl]-4-hydroxymethylphenyl isobutyrate, is a new, potent and competitive muscarinic antagonist and useful in the potential treatment of urinary incontinence. Fesoterodine is represented by the following structural formula I:

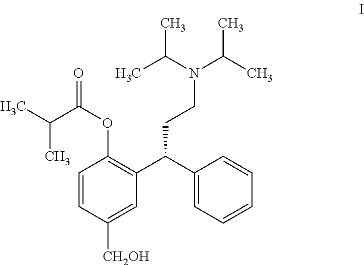

Processes for the preparation of fesoterodine and its related compounds, and their pharmaceutically acceptable salts are disclosed in U.S. Pat. Nos. 6,713,464 and 6,858,650; U.S. Patent Application No. 2006/0270738 and PCT Publication No. WO 2007/138440.

According to the U.S. Pat. No. 6,713,464 B1 (herein after referred to as the '464 patent), fesoterodine is prepared by the reaction of (±)-6-bromo-4-phenylchroman-2-one with benzyl chloride in the presence of sodium iodide and anhydrous potassium carbonate in methanol and acetone to produce (±)-3-(2-benzyloxy-5-bromophenyl)-3-phenylpropionic acid methyl ester as a light yellow oil. This product is reduced with lithium aluminium hydride in tetrahydrofuran at room temperature (reaction time: 18 hours) to produce (±)-3-(2-benzyloxy-5-bromophenyl)-3-phenylpropan-1-ol, which is then treated with p-toluenesulphonyl chloride in the presence of pyridine in dichloromethane to afford (±)-toluene-4-sulphonic acid 3-(2-benz yloxy-5-bromophenyl)-3-phenylpropyl ester. This product is then reacted with N,N-diisopropylamine in acetonitrile at reflux temperature (i.e., 75-80° C.) for 97 hours to produce (±)-[3-(2-benzyloxy-5-bromophenyl)-3-phenylpropyl]-diisopropylamine as a brown and viscous syrup. This product is resolved to produce (R)-[3-(2-benzyloxy-5-bromophenyl)-3-phenylpropyl]-diisopropylamine, which is thin subjected to Grignard reaction with ethylbromide and magnesium in the presence of solid carbon dioxide tetrahydrofuran produce (R)-4-benzyloxy-3-(3-diisopropylamino-1-phenylpropyl)-benzoic acid hydrochloride, followed by esterification with methanol in the presence of sulphuric acid to produce (R)-4-benzyloxy-3-(3-diisopropylamino-1-phenylpropyl)-benzoic acid methyl ester. This product is reduced with lithium aluminium hydride (reaction time: 18 hours) to produce (R)-[4-benzyloxy-3-(3-diisopropylamino-1-phenylpropyl)-phenyl]-methanol, which is then subjected to deprotection with Raney Nickel to produce (R)-2-(3-diisopropylamino-1-phenylpropyl)-4-hydroxymethylphenol, followed by condensation with isobutyryl chloride in an inert solvent in the presence of a base to produce fesoterodine.

While the '464 patent mentions that some of the disclosed compounds which can form a salt with physiologically acceptable organic and inorganic acids such as hydrochloride and hydrobromide, only the hydrochloride salts of the disclosed compounds have been prepared.

U.S. Pat. No. 6,858,650 (herein after referred to as the '650 patent) describes various acid addition salts of 3,3-diphenylpropylamine derivatives such as hydrochloric acid, hydrobromic acid, phosphoric acid, sulphuric acid, nitric acid, acetic acid, propionic acid, palmitic acid, stearic acid, maleic acid, fumaric acid, oxalic acid, succinic acid. DL-malic acid, L-(−)-malic acid, D-(+)-malic acid, DL-tartaric acid, L-(+)-tartaric acid, D-(−)-tartaric acid, citric acid, L-aspartic acid, L-(+)-ascorbic acid, D-(+)-glucuronic acid, 2-oxopropionic acid (pyruvic acid), furan-2-carboxylic acid (mucic acid), benzoic acid, 4-hydroxybenzoic acid, salicyclic acid, vanillic acid, 4-hydroxycinammic acid, gallic acid, hippuric acid, aceturic acid, phloretinic acid, phthalic acid, methanesulfonic acid or orotic acid. Although the '650 patent teaches several physiologically compatible acid addition salts, only hydrogen fumarate and hydrochloride hydrate salts of the disclosed compounds have been prepared.

There remains a need for new addition salts of fesoterodine.

### SUMMARY

The mandelate salt of fesoterodine has not been reported, isolated, or characterized in the literature. The present inventors have surprisingly and unexpectedly found that mandelate salt of 2-[(1R)-3-[bis(1-methylethyl)amino]1-phenylpropyl]-4-hydroxymethylphenyl isobutyrate, i.e., fesoterodine mandelate, can be isolated in a pure solid state form.

It has also been found that the solid state form of fesoterodine mandelate is useful intermediate in the preparation of fesoterodine free base, or a pharmaceutically acceptable salt thereof, preferably fesoterodine fumarate, in high purity. The mandelate salt of fesoterodine has good flow properties and is far more stable than the prior art salts at room temperature, enhanced temperature, at relative high humidities, in aqueous media. The novel mandelate salt is suitable for formulating fesoterodine.

In one aspect, a novel mandelate salt of fesoterodine is provided. In an aspect, fesoterodine mandelate in a solid state form is provided.

In another aspect, the solid state form of fesoterodine mandelate exists in an amorphous form or a crystalline form. In yet another aspect, the solid state form of fesoterodine mandelate exists in an anhydrous and/or solvent-free form or as a hydrate and/or a solvate form.

In another aspect, encompassed herein is a process for preparing the fesoterodine mandelate salt comprising contacting fesoterodine free base with mandelic acid in a suitable solvent under suitable conditions, and isolating the fesoterodine mandelate as a solid.

In another aspect, encompassed herein is a process for preparing substantially pure fesoterodine free base or a pharmaceutically acceptable salt thereof by using the solid state form of fesoterodine mandelate salt disclosed herein.

In another aspect, provided herein is a method for treating a patient suffering from diseases caused by urinary incontinence, gastrointestinal hyperactivity (irritable bowel syndrome) and other smooth muscle contractile conditions, comprising administering the solid state form of fesoterodine mandelate, or a pharmaceutical composition that comprises the solid state form of fesoterodine mandelate along with pharmaceutically acceptable excipients.

In another aspect, provided herein is a pharmaceutical composition comprising solid state form of fesoterodine mandelate, and one or more pharmaceutically acceptable excipients.

In still another aspect, provided herein is a pharmaceutical composition comprising a solid state form of fesoterodine mandelate made by the process disclosed herein, and one or more pharmaceutically acceptable excipients.

In still further aspect, encompassed is a process for preparing a pharmaceutical formulation comprising combining a solid state form of fesoterodine mandelate with one or more pharmaceutically acceptable excipients.

In another aspect, the solid state form of fesoterodine intimidate disclosed herein for use in the pharmaceutical compositions has a 90 volume-percent of the particles (D₉₀) having a size of less than or equal to about 500 microns, specifically less than or equal to about 300 microns, more specifically less than or equal to about 100 microns, still more specifically less than or equal to about 60 microns, and most specifically less than or equal to about 15 microns.

### BRIEF DESCRIPTION OF THE DRAWINGS

FIG. 1 is a characteristic powder X-ray diffraction (XRD) pattern of fesoterodine mandelate.

FIG. 2 is a characteristic differential scanning calorimetric (DSC) thermogram of fesoterodine mandelate.

FIG. 3 is a characteristic infrared (IR) spectrum fesoterodine mandelate.

### DETAILED DESCRIPTION

Disclosed herein is the unexpected discovery that fesoterodine mandelate can be obtained as a solid.

In the formulation of drug compositions, it is important for the active pharmaceutical ingredient to be in a form in which it can be conveniently handled and processed. Convenient handling is important not only from the perspective of obtaining a commercially viable manufacturing process, but also from the perspective of subsequent manufacture of pharmaceutical formulations (e.g., oral dosage forms such as tablets) comprising the active pharmaceutical ingredient.

Chemical stability, solid state stability, and “shelf life” of the active pharmaceutical ingredient are important properties for a pharmaceutically active compound. The active pharmaceutical ingredient, and compositions containing it, should be capable of being effectively stored over appreciable periods of time, without exhibiting a significant change in the physico-chemical characteristics of the active pharmaceutical ingredient, e.g., its chemical composition, density, hygroscopicity and solubility. Thus, in the manufacture of commercially viable and pharmaceutically acceptable drug compositions, it is important, wherever possible, to provide the active pharmaceutical ingredient in a stable form.

New salt forms of a pharmaceutical agent can further the development of formulation; for the treatment of illnesses. For instance, solid forms of a compound are known in the pharmaceutical arts to affect, for example, the solubility, dissolution rate, bioavailability, chemical and physical stability, flowability, tractability, and compressibility of the compound, as well as the safety and efficacy of drug products based on the compound.

The discovery of novel salts in solid state forms, including amorphous and crystalline forms, of pharmaceutically useful compound provides a new opportunity to improve the performance characteristics of a pharmaceutical product. It also adds value to the material that a formulation scientist can use the same for designing, for example, a pharmaceutical dosage form of a drug with a targeted release profile or other desired characteristic.

A novel mandelate salt of fesoterodine and its solid state forms have now been discovered.

The mandelate salt of fesoterodine has the following advantages when compared to the fumarate salt:
i) during filtration, the fumarate salt absorbs moisture, while mandelate does not absorb moisture; and
ii) isolation of the mandelate salt is very easy and it takes a shorter time period; alcoholic solvents can be used for isolation, whereas the isolation of fumarate salt involves the use of a solvent mixture containing alcohol and diisopropyl ether, and during isolation, the fumarate salt initially separates as a sticky mass and then slowly becomes solid over a period.

According to one aspect, provided herein is a novel mandelate salt of 2-[(1R)-3-[bis(1-methylethyl)amino]-1-phenylpropyl]-4-hydroxymethylphenyl isobutyrate, i.e., fesoterodine mandelate.

According to another aspect, there is provided a novel and stable solid state form of fesoterodine mandelate.

In one embodiment, the solid state form of fesoterodine mandelate is an amorphous form or a crystalline form.

In another embodiment, the solid state form of fesoterodine mandelate exists in an anhydrous and/or solvent-free form or as a hydrate and/or a solvate form. Such solvated or hydrated forms may be present as hemi-, mono-, sesqui-, di- or tri-solvates or hydrates. Solvates and hydrates may be formed as a result of solvents used during the formation of the fesoterodine mandelate becoming imbedded in the solid lattice structure. Because formation of the solvates and hydrates occurs during the preparation of fesoterodine mandelate, formation of a particular solvated or hydrated form depends greatly on the conditions and method used to prepare the salt. Solvents should be pharmaceutically acceptable.

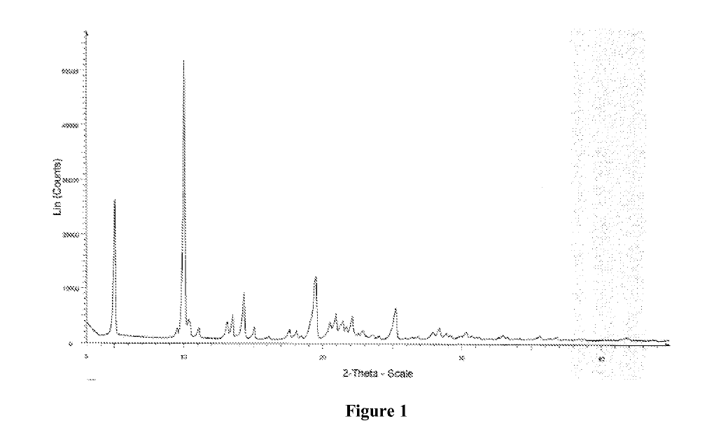

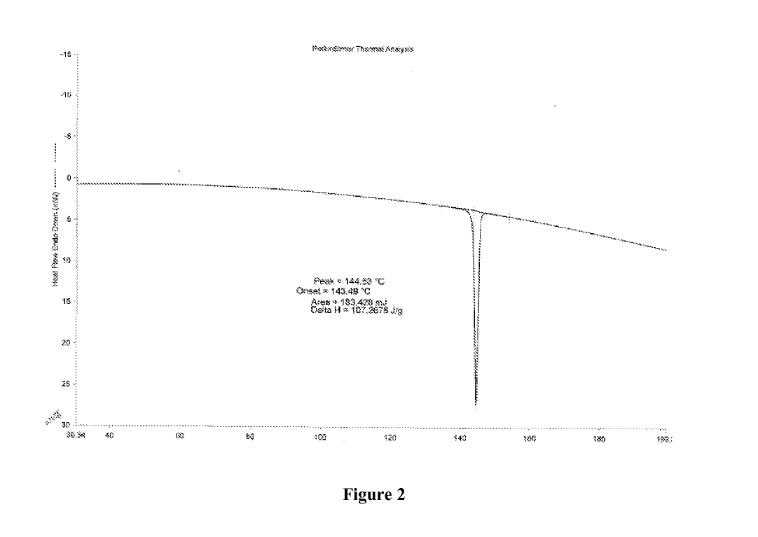

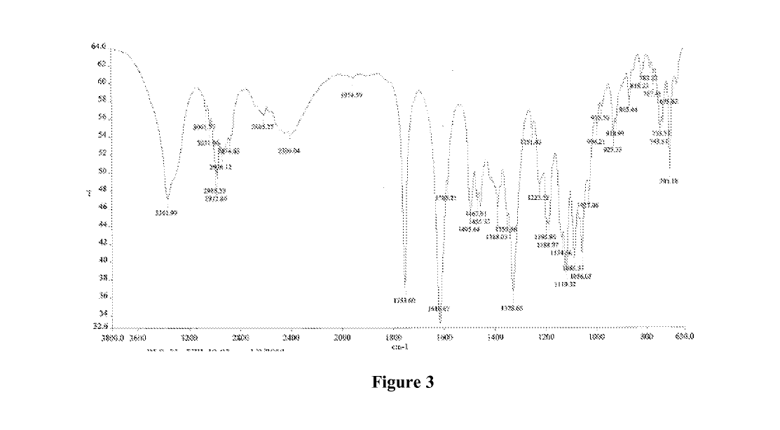

According to another aspect, the solid state form of fesoterodine mandelate is characterized by at least one, or more, of the following properties:
i) a powder X-ray diffraction pattern substantially in accordance with FIG. 1;
ii) a powder X-ray diffraction pattern having peaks at about 4.98, 10.0, 14.29, 19.46 and 25.22±0.2 degrees 2-theta substantially as depicted in FIG. 1;
iii) a powder X-ray diffraction pattern having additional peaks at about 9.49, 10.33, 11.03, 13.10, 13.47, 15.04, 17.56, 20.51, 20.92, 21.40, 22.09 and 28.37±0.2 degrees 2-theta substantially as depicted in FIG. 1;
iv) a DSC thermogram having endotherm peak at about 144° C. substantially as depicted in FIG. 2;
v) an ER spectrum substantially in accordance with FIG. 3; and/or
vi) an IR spectrum having absorption bands at about 3361, 2985, 2972, 2399, 1753, 1616, 1495, 1388, 1328, 1195, 1188, 1134, 1119, 1056, 1027, 929, 918, 733, 743 and 705±2 cm⁻¹.

The solid fesoterodine mandelate is stable, consistently reproducible, and is particularly suitable for bulk preparation and handling. Moreover, the solid state form of fesoterodine mandelate is a useful intermediate in the preparation of fesoterodine free base or a pharmaceutically acceptable salt thereof, preferably fesoterodine fumarate, in high purity.

The solid state form of fesoterodine mandelate has good flow properties and is far more stable than the prior art salts at room temperature, enhanced temperature, at relative high humidities, and in aqueous media. The novel solid state form of fesoterodine mandelate is suitable for formulating fesoterodine.

According to another aspect, there is provided a process for the preparation of fesoterodine mandelate salt, comprising:
a) providing a first solution of fesoterodine free base in an organic solvent;
b) combining the first solution with mandelic acid to produce a second solution containing fesoterodine mandelate; and
c) rig solid state form of fesoterodine mandelate from the second solution.

The fesoterodine mandelate obtained by the process disclosed herein is optionally converted into fesoterodine free base or a pharmaceutically acceptable salt thereof.

The process can produce solid state form of fesoterodine mandelate in substantially pure form.

The term “substantially pure solid state form of fesoterodine mandelate” refers to the solid state abrin of fesoterodine mandelate having a purity of greater than about 99 wt %, specifically greater than about 99.5 wt %, more specifically greater than about 99.8 wt %, and still more specifically greater than about 99.9 wt %. The purity is preferably measured by High Performance Liquid Chromatography (HPLC). For example, the purity of solid state form of fesoterodine mandelate obtained by the process disclosed herein can be about 99% to about 99.95%, or about 99.5% to about 99.99%, as measured by HPLC.

In one embodiment, the process disclosed herein provides stable crystalline form of fesoterodine mandelate. The term “stable crystalline form” refers to stability of the crystalline form under the standard temperature and humidity conditions of testing of pharmaceutical products, wherein the stability is indicated by preservation of the original polymorphic form.

Exemplary organic solvents used in step-(a) include, but are not limited to, alcohols, ketones, chlorinated hydrocarbons, esters, nitriles, polar aprotic solvents, and mixtures thereof. The term solvent also includes mixtures of solvents.

In one embodiment, the organic solvent is selected from the group consisting of methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate; methylene chloride, ethylene dichloride, chloroform, N,N-dimethylformamide, N,N-dimethylacetamide, dimethylsulfoxide, and mixtures thereof; more specifically the solvent is selected from the group consisting of methanol, ethanol, isopropyl alcohol, acetone, and mixtures thereof; and most specifically the solvent is isopropyl alcohol.

Step-(a) of providing a first solution of fesoterodine free base includes dissolving fesoterodine tree base in the organic solvent, or obtaining an existing solution from a previous processing step.

In one embodiment, the fesoterodine free base is dissolved in the organic solvent at a temperature of below boiling temperature of the solvent used specifically at about 30° C. to about 110° C., and more specifically at about 40° C. to about 80° C.

In another embodiment, the first solution in step-(a) is prepared by reacting (R)-2-(3-diisopropylamino-1-phenylpropyl)-4-hydroxymethylphenol with isobutyryl chloride in a reaction inert solvent under suitable conditions to produce a reaction mass containing crude fesoterodine free base, followed by usual work up such as washings, extractions, evaporations, etc. In one embodiment, the work-up includes dissolving or extracting the resulting fesoterodine free base residue in the organic solvent at a temperature of below boiling temperature of the solvent used, specifically at about 30° C. to about 110° C., and more specifically at about 40° C. to about 80° C.

Exemplary reaction inert solvents suitable for facilitating the reaction between (R)-2-(3-diisopropylamino-1-phenylpropyl)-4-hydroxymethylphenol and isobutyryl chloride include, but are not limited to, water, alcohols, ketones, cyclic ethers, aliphatic ethers, hydrocarbons, chlorinated hydrocarbons, nitriles, esters, polar aprotic solvents, and the like, and mixtures thereof. In one embodiment, the solvent is selected from the group consisting of water, methanol, ethanol, n-propanol, isopropanol, n-butanol, isobutanol, tort-butanol, amyl alcohol, hexanol, acetone, methyl ethyl ketone, methyl isobutyl ketone, methyl tertbutyl ketone, acetonitrile, ethyl acetate, methyl acetate, isopropyl acetate, tert-butyl methyl acetate, ethyl formate, methylene chloride, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethyl formamide, N,N-dimethylacetamide, dimethylsulfoxide, and mixtures thereof. A specific reaction inert solvent is methylene chloride.

Alternatively, the first solution in step-(a) is prepared by treating an acid addition salt of fesoterodine with a base to liberate fesoterodine free base, followed by extracting or dissolving the fesoterodine free base in the organic solvent at a temperature of below boiling temperature of the solvent used, specifically at about 30° C. to about 110° C., and more specifically at about 40° C. to about 80° C.

In another embodiment, the acid addition salt of fesoterodine is derived from a therapeutically acceptable acid such as hydrochloric acid, hydrobromic acid, acetic acid, propionic acid, sulfuric acid, nitric acid, phosphoric acid, succinic acid, maleic acid, fumaric acid, citric acid, glutaric acid, citraconic acid, glutaconic acid, tartaric acid, maleic acid, and ascorbic acid. A specific acid addition salt is fesoterodine fumarate.

The treatment of an acid addition salt with a base is carried out in a solvent. A wide variety of solvents such as chlorinated solvents, alcohols, ketones, hydrocarbon solvents, esters, other solvents etc., can be used.

In one embodiment, the base is an organic or inorganic base. Specific organic bases are triethyl amine, dimethyl amine and tert-butyl amine.

In another embodiment, the base is an inorganic base. Exemplary inorganic bases include, but are not limited to, aqueous ammonia; hydroxides, carbonates and bicarbonates of alkali or alkaline earth metals. Specific inorganic bases are aqueous ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically sodium hydroxide, potassium hydroxide, sodium carbonate and potassium carbonate.

The first solution obtained in step-(a) is optionally stirred at a temperature of about 30° C. to the reflux temperature of the solvent used for at least 20 minutes, and specifically at a temperature of about 40° C. to the reflux temperature of the solvent used for about 30 minutes to about 10 hours.

The first solution obtained in step-(a) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing fesoterodine free base by removing charcoal or silica gel. Preferably, finely powdered carbon is an active carbon. A specific mesh size of silica gel is 40-500 mesh, and more specifically 60-120 mesh.

In one embodiment, the mandelic acid used in step-(b) is an optically active mandelic acid, i.e., L-(+)-mandelic acid or D-(−)-mandelic acid. A specific optically active mandelic acid is L-(+)-mandelic acid.

In another embodiment, the mandelic acid used in step-(b) in the molar ratio of about 0.85 to 1.2 moles, specifically about 0.95 to 1.05 moles, per mole of fesoterodine free base.

Combining of the first solution with mandelic acid in step-(b) is done in a suitable order, for example, the first solution is added to the mandelic acid, or alternatively, the mandelic acid is added to the first solution. The addition is, for example, carried out drop wise or in one portion or in more than one portion. The addition is specifically carried out at a temperature of about 30° C. to about 100° C., more specifically at about 40° C. to about 90° C., and most specifically at about 40° C. to about 80° C. under stirring. After completion of addition process, the resulting mass is stirred at a temperature of about 30° C. to about 100° C. for at least 10 minutes and specifically at a temperature of about 4° C. to about 80° C. for about 30 minutes to about 8 hours to produce a second solution.

The second solution obtained in step-(b) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing fesoterodine mandelate by removing charcoal or silica gel. Preferably, finely powdered carbon is an active carbon. A specific mesh size of silica gel is 40-500 mesh, and more specifically 60-120 mesh.

The second solution obtained in step-(b) is optionally cooled at a temperature of about 20° C. to about 40° C., and specifically at a temperature of about 25° C. to about 30° C. while stirring. In one embodiment, the stirring is performed for at least about 30 minutes, and specifically for about 30 minutes to about 10 hours.

The isolation of pure fesoterodine mandelate in step-(c) is carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

In one embodiment, the crystallization is carried out by cooling the solution at a temperature of below 20° C. for at least 15 minutes, specifically at about 0° C. to about 20° C. for about 30 minutes to about 20 hours, and more specifically at about 5° C. to about 15° C. for about 1 hour to about 8 hours.

The substantially pure solid state form of fesoterodine mandelate obtained in step-(c) may be recovered by methods such as filtration, filtration under vacuum, decantation, centrifugation, or a combination thereof. In one embodiment, solid state form of fesoterodine mandelate is isolated by filtration employing a filtration media of, for example, a silica gel or celite.

The pure solid state form of fesoterodine mandelate obtained by above process may be further dried in, for example, a Vacuum Tray Dryer, Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, temperatures such as about 35° C. to about 70° C. The drying can be carried out for any desired time period that achieves the desired result, such as about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The purity of the fesoterodine mandelate obtained by the process disclosed herein is greater than about 99%, specifically greater than about 99.5%, more specifically greater than about 99.9%, and most specifically greater than about 99.95% as measured by HPLC. For example, the purity of the fesoterodine mandelate can be about 99% to about 99.95%, or about 99.5% to about 99.9%.

Fesoterodine and pharmaceutically acceptable salts of fesoterodine can be prepared in high purity by using the substantially pure fesoterodine mandelate obtained according to the process disclosed herein.

According to another aspect, there is provided a process for preparing substantially pure fesoterodine free base or as pharmaceutically acceptable salt thereof, comprising:
a) contacting fesoterodine mandelate with a base and/or an acid in a solvent to provide a reaction mass containing fesoterodine free base or a pharmaceutically acceptable salt thereof; and
b) isolating highly pure fesoterodine free base or a pharmaceutically acceptable salt thereof from the reaction mass.

Exemplary solvents used in step-(a) include, but are not limited to, water, alcohols, ketones, chlorinated hydrocarbons, hydrocarbons, nitriles, esters, ethers, polar aprotic solvents, and mixtures thereof. The term solvent includes mixtures of solvents.

In one embodiment, the solvent is selected from the group consisting of water, methanol, ethanol, n-propanol, isopropyl alcohol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; more specifically the solvent is selected from the group consisting of water, methanol, ethanol, isopropyl alcohol, acetone, methyl ethyl ketone, methyl isobutyl ketone, methylene chloride, n-pentane, n-hexane, n-heptane, cyclohexane, toluene, diethyl ether, diisopropyl ether and mixtures thereof; and most specifically the solvent is selected from the group consisting of water, methanol, ethanol, isopropyl alcohol, acetone, methyl ethyl ketone, cyclohexane, diisopropyl ether, and mixtures thereof.

In one embodiment, the base used in step-(a) is an organic or inorganic base. Exemplary organic bases include, but are not limited to, triethyl amine, dimethyl amine and tert-butyl amine.

In another embodiment, the base is an inorganic base. Exemplary inorganic bases include, but are not limited to, aqueous ammonia; hydroxides, carbonates and bicarbonates of alkali or alkaline earth metals. Specific inorganic bases are aqueous ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically sodium hydroxide, potassium hydroxide, sodium carbonate and potassium carbonate.

If the reaction in step-(a) is carried out in the presence of a base, the product obtained fesoterodine base, which can be converted in-situ into a pharmaceutically acceptable acid addition salt of fesoterodine using a suitable acid in a suitable solvent. In one embodiment, the pharmaceutically acceptable acid addition salts of fesoterodine can be obtained directly in step-(a) by carrying out the reaction in the presence of a suitable acid.

Exemplary acids used in step-(a) include, but are not limited to, organic and inorganic acids, for example, hydrochloric acid, hydrobromic acid, hydroiodic acid, acetic acid, oxalic acid, fumaric acid, maleic acid, tartaric acid, di-p-toluoyl-L-(+)-tartaric acid, succinic acid, benzenesulfonic acid, toluenesulfonic acid and methanesulfonic acid; and more preferable acids are hydrochloric acid and fumaric acid.

The reaction in step-(a) is carried out at a temperature of −25° C. to the reflux temperature of the solvent used, specifically at a temperature of 0° C. to the reflux temperature of the solvent used, more specifically at a temperature of 25° C. to the reflux temperature of the solvent used, and most specifically at the reflux temperature of the solvent used.

As used herein, “reflux temperature” means the temperature at which the solvent or solvent system refluxes or boils at atmospheric pressure.

The reaction mass containing the pure fesoterodine or a pharmaceutically acceptable salt thereof may be subjected to usual work up such as washings, extractions, evaporations etc., followed by isolation from a suitable organic solvent by methods known in the art.

The isolation of highly pure fesoterodine or a pharmaceutically acceptable salt thereof in stop-(b) is carried out by forcible or spontaneous crystallization.

In one embodiment, the crystallization is carried out by cooling the solution at a temperature of below 30° C. for at least 15 minutes, specifically at about 0° C. to about 30° C. for about 30 minutes to about 20 hours, and more specifically at about 0° C. to about 25° C. for about 1 hours to about 5 hours.

The pure fesoterodine or a pharmaceutically acceptable salt thereof obtained by above process is recovered and optionally further dried as described above.

Exemplary pharmaceutically acceptable salts of fesoterodine include hydrochloride, hydrobromide, sulfate, fumarate and tartarate, and more preferably fumarate.

The purity of the fesoterodine or a pharmaceutically acceptable salt thereof obtained by the process disclosed herein is of greater than about 99%, specifically greater than about 99.5%, more specifically greater than about 99.9%, and most specifically greater than about 99.95% as measured by HPLC. For example, the purity of the fesoterodine or a pharmaceutically acceptable salt thereof can be about 99% to about 99.95%, or about 99.5% to about 99.99%.

Further encompassed herein is the use of the solid state form of fesoterodine mandelate for the manufacture of a pharmaceutical composition together with a pharmaceutically acceptable carrier.

A specific pharmaceutical composition of the solid state fort of fesoterodine mandelate is selected from a solid dosage form and an oral suspension.

In one embodiment, the solid state form of fesoterodine mandelate has a D₉₀ particle size of less than or equal to about 500 microns, specifically less than or equal to about 300 microns, more specifically less than or equal to about 100 microns, still more specifically less than or equal to about 60 microns, and most specifically less than or equal to about 15 microns.

In another embodiment, the particle sizes of the solid state form of fesoterodine mandelate are produced by a mechanical process of reducing the size of particles which includes any one or more of cutting, chipping, crushing, milling, grinding, micronizing, trituration or other particle size reduction methods known in the art, to bring the solid state corm to the desired particle size range.

According to another aspect, there is provided pharmaceutical compositions comprising solid state limn of fesoterodine mandelate salt and one or more pharmaceutically acceptable excipients.

According to another aspect, there is provided pharmaceutical compositions comprising the solid state form of fesoterodine mandelate prepared according to processes disclosed herein and one or more pharmaceutically acceptable excipients.

According to another aspect, there is provided a process for preparing a pharmaceutical formulation comprising combining the solid state form of fesoterodine mandelate prepared according to processes disclosed herein, with one or more pharmaceutically acceptable excipients.

According to another aspect, there is provided a method for treating a patient suffering from diseases caused by urinary incontinence, gastrointestinal hyperactivity (irritable bowel syndrome) and other smooth muscle contractile conditions, comprising administering the solid state form of fesoterodine mandelate, or a pharmaceutical composition that comprises the solid state form of fesoterodine mandelate along with pharmaceutically acceptable excipients.

Yet in another embodiment, pharmaceutical compositions comprise at least a therapeutically effective amount of solid state form of fesoterodine mandelate. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to tablets, pills, capsules, syrup, troches, sachets, suspensions, powders, lozenges, elixirs and the like. The solid state form of fesoterodine mandelate may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain solid state form of fesoterodine mandelate within a capsule which may be coated with gelatin. Tablets and powders may also be coated with an enteric coating. Suitable enteric coating include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising it powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Instrumental Details:

### X-Ray Powder Diffraction (P-XRD):

The X-Ray powder diffraction was measured by an X-ray powder Diffractometer equipped with CuKα-radiations (40 kV, 40 mA) in wide-angle X-ray Diffractometer of BRUKER axs, D8 ADVANCE. The sample was analyzed using the following instrument parameters: measuring range 3-45° 2-theta; step width=0.01579°; and measuring time per step=0.11 sec.

### Differential Scanning Calorimetry (DSC)

DSC (Differential Scanning calorimetry) measurements were performed with a Differential Scanning calorimeter (Diamond DSC, Perkin-Elmer) at a scan rate of 5° C. per minute. The nitrogen gas purge was at 40 ml/min. The instrument was calibrated for temperature and heat flow using indium as standards. The samples were encapsulated in to dosed aluminium pans without hole subsequently crimped to ensure a tight seal. Data acquisition and analysis were performed using pyris software.

### Infra-Red Spectroscopy (FT-IR):

FT-IR spectroscopy was carried out with a Perkin Elmer Spectrum 100 series spectrometer. For the production of the KBr compacts approximately 2 mg of sample was powdered with 200 mg of KBr. The spectra were recorded in transmission mode ranging from 3800 to 650 cm⁻¹.

The following examples are given for the purpose of illustrating the present disclosure and should not be considered as limitation on the scope or spirit of the disclosure.

### EXAMPLES

### Example 1

### Preparation of Fesoterodine Mandelate

(R)-2-(3-Diisopropylamino-1-phenylpropyl)-4-hydroxymethylphenol (100 g) was added to methylene chloride (1200 ml) at 25-30° C. under stirring and the resulting mass was cooled to −10° C. A solution of isobutyryl chloride (33 g) in methylene chloride (800 ml) was added drop wise to the above mass at −10° C. to −5° C. over a period of 1 hour and the resulting mass was stirred for 30 minutes at the same temperature. The temperature of the mass was raised to 0° C. followed by the addition of water (400 ml). The resulting mass was stirred for 5 minutes. The organic layer was separated followed by the addition of aqueous sodium bicarbonate solution (28.5 g in 400 ml water) and then stirred for 15 minutes. The resulting organic layer was separated and washed with water (500 ml). The organic layer was distilled completely under vacuum followed by the addition of isopropyl alcohol (500 ml). The temperature of the resulting mass was raised to 55° C. followed by the addition of L-(+)-mandelic acid (42.5 g) and stirred for 30 minutes at the same temperature. The resulting mass was cooled to 25-30° C. and then stirred for 6 hours at the same temperature. The resulting mass was further cooled 10-15° C. and stirred for 1 hour. The precipitated solid was filtered, washed with chilled isopropyl alcohol (100 ml) and then dried the product under vacuum at 50° C. to produce 85 g of fesoterodine mandelate. (Purity by HPLC: 99.72%).

### Example 2

### Preparation of Fesoterodine Fumarate

Fesoterodine mandelate (100 g, obtained according to example) was added to methylene chloride (500 ml) at 25-30° C. The resulting mixture was stirred for 10 minutes and then washed with 10% sodium hydroxide solution (200 ml). The resulting layers were separated and the organic layer was washed with water (200 ml). The organic layer was separated and dried over sodium sulfate (10 g) followed by distillation of methylene chloride under vacuum to produce fesoterodine as oily mass. This oily mass was followed by the addition of methyl ethyl ketone (170 ml) at 25-30° C., stirred for 10 minutes and then heated to 80° C. Fumaric acid (21.5 g) was added to the resulting mass, stirred for 1 hour at 80° C. followed, by the drop wise addition of cyclohexane (70 ml) at 80° C. and stirred for 1 hour. The resulting mass was slowly cooled to 25-30° C. and stirred for 12 hours at the same temperature. The resulting mass was then cooled to 0-5° C. and stirred for 12 hours at 0-5° C. The separated solid was filtered, washed with the mixture of cyclohexane (135 ml) and methyl ethyl ketone (15 ml), and then dried under vacuum at 45-50° C. to produce 80 g of fesoterodine fumarate (Purity by HPLC: 99.76%)

### Example 3

### Preparation of Fesoterodine Fumarate

Fesoterodine mandelate (100 g, obtained according to example 1) was added to methylene chloride (500 ml) at 25-30° C. The resulting mixture was stirred for 10 minutes and then washed with aqueous sodium carbonate solution (28 g in 500 ml of water). The resulting layers were separated and the organic layer was washed with water (200 ml). The organic layer was separated and dried over sodium sulfate (10 g) followed by distillation of methylene chloride under vacuum to produce fesoterodine as oily mass. This oily mass was followed by the addition of isopropyl alcohol (250 ml) at 25-30° C., stirred for 10 minutes and then heated to 55-60° C. Fumaric acid (20.6 g) was added to the resulting mass and stirred for 30 minutes at 35-60° C. The resulting mass was cooled to 25-30° C. followed by the dropwise addition of diisopropyl ether (900 ml) at 25-30° C. and then stirred for 12 hours at the same temperature. The separated solid was filtered, with diisopropyl ether (300 ml) and then dried under vacuum at 45-50° C. to produce 84 g of fesoterodine fumarate (Purity by HPLC: 99.86%)

### Example 4

### Stability of Solid State Form of Fesoterodine Mandelate

Fesoterodine mandelate was prepared according to the process exemplified in Example 1 and was packed in a self-sealing low-density polyethylene (LDPE) bag. The material was stored for 3 months under normal atmospheric conditions at room temperature and checked for polymorphic stability.

The material was found to retain its polymorphic form after three months of holding, as indicated by maintenance of the original P-XRD pattern.

Unless otherwise indicated, the following definitions are set forth to illustrate and define the meaning and scope of the various terms used to describe the invention herein.

The term “fesoterodine mandelate”, as used herein, means a salt comprised of fesoterodine cations and mandelate anions. For instance, solid as well as dissolved forms are included, and so are crystalline and amorphous forms. Fesoterodine mandelate may exist in an anhydrous and/or solvent-free form or as a hydrate and/or a solvate.

Further, the term “fesoterodine mandelate”, as used herein, encompasses stoichiometric as well as non-stoichiometric ratios of fesoterodine cations and mandelate anions. In a particular embodiment, fesoterodine mandelate is formed as a salt having a 1:1 molar ratio between fesoterodine cation and mandelate anion even when an excess of fesoterodine or an excess mandelic acid is used in the salt formation.

The term “solid form of fesoterodine mandelate disclosed herein” includes crystalline forms, amorphous forms, hydrated, and solvated forms of fesoterodine mandelate.

The term “crystalline polymorph” refers to a crystal modification that can be characterized by analytical methods such as X-ray powder diffraction, IR-spectroscopy, differential scanning calorimetry (DSC) or by its melting point.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” an as used herein means the amount of compound that, when administered to a mammal for treating a state, disorder or condition, is sufficient to of such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering, agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used (cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking, effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent.

As used herein, D_{X} means that X percent of the particles have a diameter less than a specified diameter D. Thus, a D₉₀ or (d(0.9) of less than 300 microns means that 90 volume-percent of the particles in a composition have a diameter less than 300 microns.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Solid state form of a mandelate salt of 2-[(1R)-3-[bis(1-methylethyl)amino]-1-phenylpropyl]-4-hydroxy-methylphenyl isobutyrate (fesoterodine mandelate salt).

2. (canceled)

3. The solid fesoterodine mandelate salt of claim 1, which is in a crystalline form or an amorphous form, wherein the solid state form is anhydrous and/or solvent-free form or as a hydrate and/or a solvate.

4. (canceled)

5. (canceled)

6. The fesoterodine mandelate salt of claim 1, characterized by one or more of the following properties:
i) a powder X-ray diffraction pattern substantially in accordance with FIG. 1;
ii) a powder X-ray diffraction pattern having peaks at about 4.98, 10.0, 14.29, 19.46 and 25.22±0.2 degrees 2-theta;
iii) a powder X-ray diffraction pattern having additional peaks at about 9.49, 10.33, 11.03, 13.10, 13.47, 15.04, 17.56, 20.51, 20.92, 21.40, 22.09 and 28.37±0.2 degrees 2-theta;
iv) a DSC thermogram having an endotherm peak at about 144° C. as depicted in FIG. 2;
v) an IR spectrum substantially in accordance with FIG. 3; and
vi) an IR spectrum having absorption bands at about 3361, 2985, 2972, 2399, 1753, 1616, 1495, 1388, 1328, 1195, 1188, 1134, 1119, 1056, 1027, 929, 918, 733, 743 and 705±2 cm⁻¹.

7. A process for the preparation of the fesoterodine mandelate salt of claim 1, comprising:
a) providing a first solution of fesoterodine free base in an organic solvent selected from the group consisting of alcohols, ketones, chlorinated hydrocarbons, esters, nitriles, polar aprotic solvents, and mixtures thereof;
b) combining the first solution with mandelic acid to produce a second solution containing fesoterodine mandelate; and
c) isolating the solid state form of fesoterodine mandelate from the second solution.

8. (canceled)

9. The process of claim 7, wherein the organic solvent used in step-(a) is selected from the group consisting of methanol, ethanol, n-propanol, isopropyl alcohol, isobutanol, n-butanol, tert-butanol, amyl alcohol, isoamyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, N,N-dimethylformamide, N,N-dimethylacetamide, dimethylsulfoxide, and mixtures thereof; wherein the mandelic acid used in step-(b) is L-(+)-mandelic acid or D-(−)-mandelic acid; and wherein the mandelic acid in step-(b) is used in a molar ratio of about 0.85 to 1.2 moles per mole of fesoterodine free base.

10. The process of claim 9, wherein the organic solvent is selected from the group consisting of methanol, ethanol, isopropyl alcohol, acetone, and mixtures thereof; wherein the mandelic acid is L-(+)-mandelic acid; and wherein the mandelic acid is used in a molar ratio of about 0.95 to 1.05 moles per mole of fesoterodine free base.

11. The process of claim 7, wherein the first solution in step-(a) is prepared by dissolving fesoterodine free base in the organic solvent at a temperature below a boiling temperature of the organic solvent; wherein the first solution in step-(a) is prepared by reacting (R)-2-(3-diisopropylamino-1-phenylpropyl)-4-hydroxymethylphenol with isobutyryl chloride in a reaction inert solvent to produce a reaction mass containing crude fesoterodine free base, subjecting the reaction mass to washings, extractions or evaporations, and dissolving or extracting the fesoterodine free base residue in the organic solvent at a temperature of below boiling temperature of the organic solvent; or wherein the first solution in step-(a) is prepared by treating an acid addition salt of fesoterodine with a base to produce fesoterodine free base; and extracting or dissolving the fesoterodine free base in the organic solvent at a temperature below a boiling temperature of the organic solvent.

12-14. (canceled)

15. The process of claim 7, wherein the first solution obtained in step-(a) is optionally stirred at a temperature of about 30° C. to a boiling temperature of the organic solvent for at least 20 minutes; wherein the first solution obtained in step-(a) is optionally subjected to carbon treatment or silica gel treatment; wherein the combining in step-(b) is accomplished by adding the first solution to the mandelic acid or by adding the mandelic acid to the first solution at a temperature of about 30° C. to about 100° C.; wherein the second solution obtained in step-(b) is optionally subjected to carbon treatment or silica gel treatment; wherein the second solution obtained in step-(b) is optionally cooled at a temperature of about 20° C. to about 40° C. for about 30 minutes to about 10 hours; and wherein the isolation in step-(c) is initiated by cooling, seeding, partial removal of the solvent from the solution, by adding an anti-solvent to the solution, or a combination thereof.

16. The process of claim 15, wherein the second solution obtained in step-(b) is stirred at a temperature of about 40° C. to a reflux temperature of the organic solvent for about 30 minutes to about 10 hours; wherein the isolation in step-(c) is carried out by cooling the solution at about 0° C. to about 20° C. for about 30 minutes to about 20 hours; wherein the solid obtained in step-(c) is recovered by filtration, filtration under vacuum, decantation, centrifugation, filtration employing a filtration media of a silica gel or celite, or a combination thereof; wherein the substantially pure solid state form of fesoterodine mandelate obtained in step-(c) is further dried under vacuum or at atmospheric pressure, at a temperature of about 35° C. to about 70° C.; and wherein the solid state form of fesoterodine mandelate obtained in step-(c) has a purity of about 99% to about 99.95% as measured by HPLC.

17-38. (canceled)

39. A process for preparing substantially pure fesoterodine or a pharmaceutically acceptable salt thereof using the solid state form of fesoterodine mandelate of claim 1, comprising:
a) contacting the solid state form of fesoterodine mandelate with a base and/or an acid in a solvent to provide a reaction mass containing fesoterodine free base or a pharmaceutically acceptable salt thereof, wherein the solvent is selected from the group consisting of water, alcohols, ketones, chlorinated hydrocarbons, hydrocarbons, nitriles, esters, ethers, polar aprotic solvents, and mixtures thereof; and
b) isolating highly pure fesoterodine free base or a pharmaceutically acceptable salt thereof from the reaction mass.

40. The process of claim 39, wherein the solvent used in step-(a) is selected from the group consisting of water, methanol, ethanol, n-propanol, isopropyl alcohol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; wherein the base used in step-(a) is an organic or inorganic base; and wherein the acid used in step-(a) is an organic or inorganic acid.

41. The process of claim 40, wherein the solvent is selected from the group consisting of water, methanol, ethanol, isopropyl alcohol, acetone, methyl ethyl ketone, cyclohexane, diisopropyl ether, and mixtures thereof; wherein the base is selected from the group consisting of triethyl amine, dimethyl amine, tert-butyl amine, aqueous ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide; and wherein the acid is selected from the group consisting of hydrochloric acid, hydrobromic acid, hydroiodic acid, acetic acid, oxalic acid, fumaric acid, maleic acid, tartaric acid, di-p-toluoyl-L-(+)-tartaric acid, succinic acid, benzenesulfonic acid, toluenesulfonic acid and methanesulfonic acid.

42-47. (canceled)

48. The process of claim 41, wherein the acid is fumaric acid.

49. The process of claim 39, wherein the reaction in step-(a) is carried out at a temperature of −25° C. to the reflux temperature of the solvent; and wherein isolation in step-(b) is carried out by forcible or spontaneous crystallization.

50. (canceled)

51. A pharmaceutical composition comprising solid state form of fesoterodine mandelate of claim 1, and one or more pharmaceutically acceptable excipients.

52. The pharmaceutical composition of claim 51, wherein the pharmaceutical composition is a solid dosage form.

53. The pharmaceutical composition of claim 51, wherein the solid state form of fesoterodine mandelate has a D₉₀ particle size of less than or equal to about 500 microns.

54. The pharmaceutical composition of claim 53, wherein the solid state form of fesoterodine mandelate has a D₉₀ particle size of less than or equal to about 300 microns; less than or equal to about 100 microns; less than or equal to about 60 microns; or less than or equal to about 15 microns.

55. A method of treating a patient suffering from diseases caused by urinary incontinence, gastrointestinal hyperactivity (irritable bowel syndrome) and other smooth muscle contractile conditions, comprising administering a pharmaceutical composition that comprises the solid state form of fesoterodine mandelate of claim 1 along with pharmaceutically acceptable excipients.
